(19) United States
(12) Patent Application Publication
(10) Pub. No.: US 2014/0350500 A1
(43) Pub. Date: Nov. 27, 2014

(54) OSTOMY GAS ELIMINATOR

(71) Applicant: Gladys Joseph, Lynn, MA (US)

(72) Inventor: Gladys Joseph, Lynn, MA (US)

(21) Appl. No.: 13/901,693

(22) Filed: May 24, 2013

(51) Int. Cl. A61F 5/441

(52) U.S. Cl. CPC A61F 5/441
USPC 604/342; 604/339

(57) ABSTRACT

An ostomy device including a bag, a lid, and a vent. The lid is replaceably attached to the bag, attaches to a site of a stoma of a patient, and self-seals to the stoma of the patient. The vent is operatively connected to the bag, and vents gas emanating from the stoma of the patient to prevent build-up of the gas that could detach the ostomy device from the site of the stoma of the patient and require rettachments that could irritate skin of the patient at the site of the stoma of the patient.

### 1. BACKGROUND OF THE INVENTION

A. Field of the Invention

The embodiments of the present invention relate to an ostomy device, and more particularly, the embodiments of the present invention relate to an ostomy device for attaching to a site of a stoma of a patient, for self-sealing to the stoma of the patient, and for venting gas emanating from the stoma of the patient to prevent build-up of the gas that could detach the ostomy device from the site of the stoma of the patient and require rettachments that could irritate skin of the patient at the site of the stoma of the patient.

B. Description of the Prior Art

An ostomy pouching system (also colloquially called a bag) is a medical device prosthetic that provides a means for the collection of waste from a surgically diverted biological system (colon, ileum, urinary) and the creation of a stoma. Pouching systems are most commonly associated with colostomies, ileostomies, and urostomies.¹

Pouching systems usually consist of a collection pouch bag,² known as a one-piece system, or in some instances, involves a mounting plate commonly called a wafer or a baseplate. A collection pouch that is attached mechanically or with an adhesive in an airtight seal is known as a two-piece system. The selection of systems varies greatly between individuals and is often based on personal preference and lifestyle.

Ostomy pouching systems collect waste that is output from a stoma. The pouching system allows the stoma to drain into a sealed collection pouch, while protecting the surrounding skin from contamination.³ ¹“Ostomy Information”. United Ostomy Associations of America. Retrieved Sep. 3, 2010.²http://www.mayoclinic.com/health/ostomy/SA00072.³Lueder, Wendy. “Ostomates' Choices”. Broward Ostomy Association. Archived from the original on 2008-11-21. Retrieved Sep. 3, 2010.

Numerous innovations for ostomy bag vents have been provided in the prior art, which will be described below in chronological order to show advancement in the art, and which are incorporated in their entirety herein by reference thereto. Even though these innovations may be suitable for the specific individual purposes to which they address, nevertheless, they differ from the present invention in that they do not teach an ostomy device for attaching to a site of a stoma of a patient, for self-sealing to the stoma of the patient, and for venting gas emanating from the stoma of the patient to prevent build-up of the gas that could detach the ostomy device from the site of the stoma of the patient and require rettachments that could irritate skin of the patient at the site of the stoma of the patient.

### (1) U.S. Pat. No. 4,451,258 to Jensen

U.S. Pat. No. 4,451,258—issued to Jensen on May 29, 1984 in U.S. class 604 and subclass 333—teaches an air vent for an ostomy bag, which has a variable flow rate so that the flow rate through a deodorizing filter housed in the vent can be varied to enhance the effectiveness of the filter. The vent also permits precise control over the degree of inflation of the bag.

### (2) U.S. Pat. No. 4,938,749 to Jensen

U.S. Pat. No. 4,938,749—issued to Jensen on Jul. 3, 1990 in U.S. class 604 and subclass 333—teaches a housing is mounted on, and sealed around, an opening in a pouch wall. A disc-like base of the housing has a central and axially extending entrance passageway aligned with the pouch opening. A portion of the base extends radially outwardly beyond the seal and has a second axial passageway therethrough. The cover of the housing defines a filter-receiving-recess that is aligned with the entrance passageway. The recess has a connecting channel extending radially therefrom. The cover is removably mounted on the base for relative rotational movement between a first position in which the connecting channel aligns with the exit passageway so as to permit venting of the gases from the pouch, and a second position wherein the connecting channel is out of alignment with the exit passageway closing the vent.

### (3) U.S. Pat. No. 5,468,235 to La Gro

U.S. Pat. No. 5,468,235—issued to La Gro on Nov. 21, 1995 in U.S. class 604 and subclass 333—teaches an ostomy pouch having a filter assembly located therein. The filter assembly includes a thin and flexible outer panel of liquid- and gas-impervious sheet material having a peripheral portion sealed to one wall of the pouch and a central portion unattached to and, preferably, spaced from that wall. The pouch wall overlying the central portion of the filter assembly's outer panel is provided with a flap vent in the form of a curvilinear slit defining at least one flap that normally assumes a closed position coplanar with surrounding portions of the wall but flexes outwardly into an open position when venting gases from the pouch. The outer panel of the filter assembly is provided with a second flap vent similar to but, preferably, out of register with the first flap vent of the pouch wall. Components of the filter assembly within the pouch function to brace the flap or flaps of the second vent against opening in any direction other than outwardly.

### (4) U.S. Pat. No. 5,470,325 to Fundock

U.S. Pat. No. 5,470,325—issued to Fundock on Nov. 28, 1995 in U.S. class 604 and subclass 332—teaches an ostomy bag including a combination venting cleaning assembly that allows gases to be exhausted from the bag through a first opening in a closure. The closure is removable so as to provide a larger opening for cleaning or flushing purposes. The bag further includes a handle attached to the tailpiece, which is grasped by the user during the cleaning process in order to avoid contact with the bodily waste products collected within the bag.

### (5) U.S. Pat. No. 5,658,267 to Colacello et al

U.S. Pat. No. 5,658,267—issued to Colacello et al on Aug. 19, 1997 in U.S. class 604 and subclass 333—teaches a manual gas vent assembly for use in venting gas from the interior of a conventional unvented ostomy collection pouch, which include a valve assembly having an open position that permits gas to pass therethrough and a closed position for preventing gas to pass therethrough. The valve assembly has an input portion for communicating with the interior of the ostomy collection pouch and an output portion for communicating with the atmosphere. A mounting device is for mounting the valve assembly to a selected portion of the wall of the ostomy collection pouch. The mounting device releasably contains therein a portion of the valve assembly so as to position the input portion thereof proximate the interior of the ostomy collection pouch. The output portion of the valve assembly is proximate to the atmosphere. Also, included is an absorbent pad suitable for receiving an antiseptic and/or deodorant.

### (6) U.S. Pat. No. 6,165,159 to Blanton

U.S. Pat. No. 6,165,159—issued to Blanton on Dec. 26, 2000 in U.S. class 604 and subclass 333—teaches a vent for a latex ostomy bag. A flexible patch is mounted over a plurality of small perforations that are formed in a wall of the bag. The side and bottom edges of the patch are attached adhesively to the surface of the bag so as to form a pouch, with the upper edge of the pouch being free from attachment so as to form an external vent opening. A piece of cotton gauze or other form of porous pad is placed in the pouch so that accumulated gas in the bag passes into the pouch through the perforations and is vented to the atmosphere via the porous pad. A liquid odor control agent is applied to the porous pad. The pad is periodically removed and replaced without having to replace the entire ostomy bag.

### (7) U.S. Pat. No. 6,328,719 to Holtermann et al

U.S. Pat. No. 6,328,719—issued to Holtermann et al. on Dec. 11, 2001 in U.S. class 604 and subclass 332—teaches a filter and gas vent system incorporated in an ostomy bag during its manufacture, which includes a filter in the shape of a flat band. Its deodorizing core is left open to the ambient air at two ends of the filter. The latter is held inside the bag by two weld seams joining walls of the bag to each other. Each of the two weld seams extends from one of two side edges of the bag to its upper edge. The filter is contained entirely in the bag, which thus isolates the ends from the outside. The material from which the core of the filter is made, often activated charcoal, no longer risks soiling the clothes or being driven off when the subject is showering.

### (8) U.S. Pat. No. 7,468,056 to Burt

U.S. Pat. No. 7,468,056—issued to Burt on Dec. 23, 2008 in U.S. class 604 and subclass 330—teaches a colostomy bag with a vent and a method for venting gas collected in the colostomy bag. A dual vent and cap assembly attached to a colostomy bag vents gas trapped in the bag either continuously or as periodically desired by a user.

It is apparent that numerous innovations for ostomy bag vents have been provided in the prior art, which are adapted to be used. Furthermore, even though these innovations may be suitable for the specific individual purposes to which they address, nevertheless, they would not be suitable for the purposes of the embodiments of the present invention as heretofore described, namely, an ostomy device for attaching to a site of a stoma of a patient, for self-sealing to the stoma of the patient, and for venting gases emanating from the stoma of the patient to prevent build-up of the gas that could detach the ostomy device from the site of the stoma of the patient and require rettachments that could irritate skin of the patient at the site of the stoma of the patient.

### 2. SUMMARY OF THE INVENTION

Thus, an object of the embodiments of the present invention is to provide an ostomy device for attaching to a site of a stoma of a patient, for self-sealing to the stoma of the patient, and for venting gas emanating from the stoma of the patient to prevent build-up of the gas that could detach the ostomy device from the site of the stoma of the patient and require rettachments that could irritate skin of the patient at the site of the stoma of the patient, which avoids the disadvantages of the prior art.

Briefly stated, another object of the embodiments of the present invention is to provide an ostomy device attached to a site of a stoma of a patient, self-sealed to the stoma of the patient, and vents gas emanating from the stoma of the patient to prevent build-up of the gas that could detach the ostomy device from the site of the stoma of the patient and require rettachments that could irritate skin of the patient at the site of the stoma of the patient. The ostomy device includes a bag, a lid, and a vent. The lid is replaceably attached to the bag, attaches to the site of the stoma of the patient, and self-seals to the stoma of the patient. The vent is operatively connected to the bag, and vents the gas emanating from the stoma of the patient to prevent the build-up of the gas that could detach the ostomy device from the site of the stoma of the patient and require rettachments that could irritate the skin of the patient at the site of the stoma of the patient.

The novel features considered characteristic of the embodiments of the present invention are set forth in the appended claims. The embodiments of the present invention themselves, however, both as to their constriction and to their method of operation together with additional objects and advantages thereof will be best understood from the following description of the specific embodiments when read and understood in connection with the accompanying figures of the drawing.

### 3. BRIEF DESCRIPTION OF THE FIGURES OF THE DRAWING

The figures of the drawing are briefly described as follows:

FIG. 1 is a diagrammatic perspective view of the ostomy device of the embodiments of the present invention fully attached to a site of a stoma of a patient, self-sealed to the stoma of the patient, and venting gas emanating from the stoma of the patient to prevent build-up of the gas that could detach the ostomy device from the site of the stoma of the patient and require rettachments that could irritate skin of the patient at the site of the stoma of the patient;

FIG. 2 is an exploded diagrammatic perspective view of the ostomy device of the embodiments of the present invention partially attached to a site of a stoma of a patient, self-sealed to the stoma of the patient, and venting gas emanating from the stoma of the patient to prevent build-up of the gas that could detach the ostomy device from the site of the stoma of the patient and require rettachments that could irritate skin of the patient at the site of the stoma of the patient;

FIG. 3 is an enlarged diagrammatic perspective view of the area generally enclosed by the dotted curve identified by ARROW 3 in FIG. 1 of the ostomy device of the embodiments of the present invention;

FIG. 4 is an enlarged and exploded diagrammatic perspective view of the area generally enclosed by the dotted curve identified by ARROW 4 in FIG. 2 of the ostomy device of the embodiments of the present invention;

FIG. 5 is an enlarged diagrammatic perspective view of the area generally enclosed by the dotted curve identified by ARROW 5 in FIG. 4 of a snap fastener of the osotomy device of the embodiments of the present invention replaceably attaching a snap-on flanged lid of the osotomy device of the embodiments of the present invention to a removable bag of the osotomy device of the embodiments of the present invention;

FIG. 6 is an enlarged diagrammatic elevational view taken generally in the direction of ARROW 6 in FIG. 4 of an openable vent of the osotomy device of the embodiments of the present invention in a closed position;

FIG. 7 is an enlarged diagrammatic elevational view taken generally in the direction of ARROW 7 in FIG. 4 of the openable vent of the osotomy device of the embodiments of the present invention in an open position;

FIG. 8 is an enlarged and exploded diagrammatic perspective view of the area generally enclosed by the dotted curve identified by ARROW 8 in FIG. 4 of a reusable and leak-proof clip of the osotomy device of the embodiments of the present invention in an open position; and

FIG. 9 is an enlarged and exploded diagrammatic perspective view of the area generally enclosed by the dotted curve identified by ARROW 9 in FIG. 4 of the reusable and leak-proof clip of the osotomy device of the embodiments of the present invention in a closed position.

### 4. LIST OF REFERENCE NUMERALS UTILIZED IN THE FIGURES OF THE DRAWING

A. Introductory.
10 ostomy device of embodiments of present invention for attaching to site 12 of stoma 14 of patient 16, for self-sealing to stoma 14 of patient 16, and for venting gas 18 emanating from stoma 14 of patient 16 to prevent build-up of gas 18 that could detach ostomy device 10 from site 12 of stoma 14 of patient 16 and require rettachments that could irritate skin 20 of patient 16 at site 12 of stoma 14 of patient 16
12 site of stoma 14 of patient 16
14 stoma of patient 16
16 patient
18 gas
20 skin of patient 16

B. Overall Configuration of Ostomy Device 10.
22 bag
24 lid for attaching to site 12 of stoma 14 of patient 16 and for self-sealing to stoma 14 of patient 16
26 vent for venting gas 18 emanating from stoma 14 of patient 16 to prevent build-up of gas 18 that could detach ostomy device 10 from site 12 of stoma 14 of patient 16 and require rettachments that could irritate skin 20 of patient 16 at site 12 of stoma 14 of patient 16
28 clip for allowing emptying of bag 22 while bag 22 is still attached to site 12 of stoma 14 of patient 16

C. Specific Configuration of Bag 22
30 wide and open top of bag 22
32 narrow and open bottom of bag 22
34 wall of bag 22
35 ring shape of wide and open top 30 of bag 22
36 rigid ring of ring shape 35 of wide and open top 30 of bag 22

D. Specific Configuration of Lid 24.
38 center of lid 24
40 perimeter of lid 24
42 through bore of lid 24 for receiving stoma 14 of patient 16
44 barrier ring of lid 24 for self-sealing to stoma 14 of patient 16 when lid 24 is attached to site 12 of stoma 14 of patient 16
46 pair of L-shaped tabs of lid 24

E. Specific Configuration of Vent 26
48 through bore of wall 34 of bag 22 of vent 26
50 collar of vent 26
52 cap of vent 26
54 hinge of vent 26
55 snap closure of vent 26
56 pair of cooperating portions of snap closure 54 of vent 26
58 recess of collar 50 of vent 26 for facilitating grabbing cap 52 of vent 26 when cap 52 of vent 26 is to be opened

F. Specific Configuration of Clip 28
60 seal of narrow and open bottom 32 of bag 22
62 pair of cooperating portions of clip 28
64 associated ends of pair of cooperating portions 62 of clip 28
66 other associated ends of pair of cooperating portions 62 of clip 28

### 5. DETAILED DESCRIPTION OF THE PREFERRED EMBODIMENTS

A. Introductory.

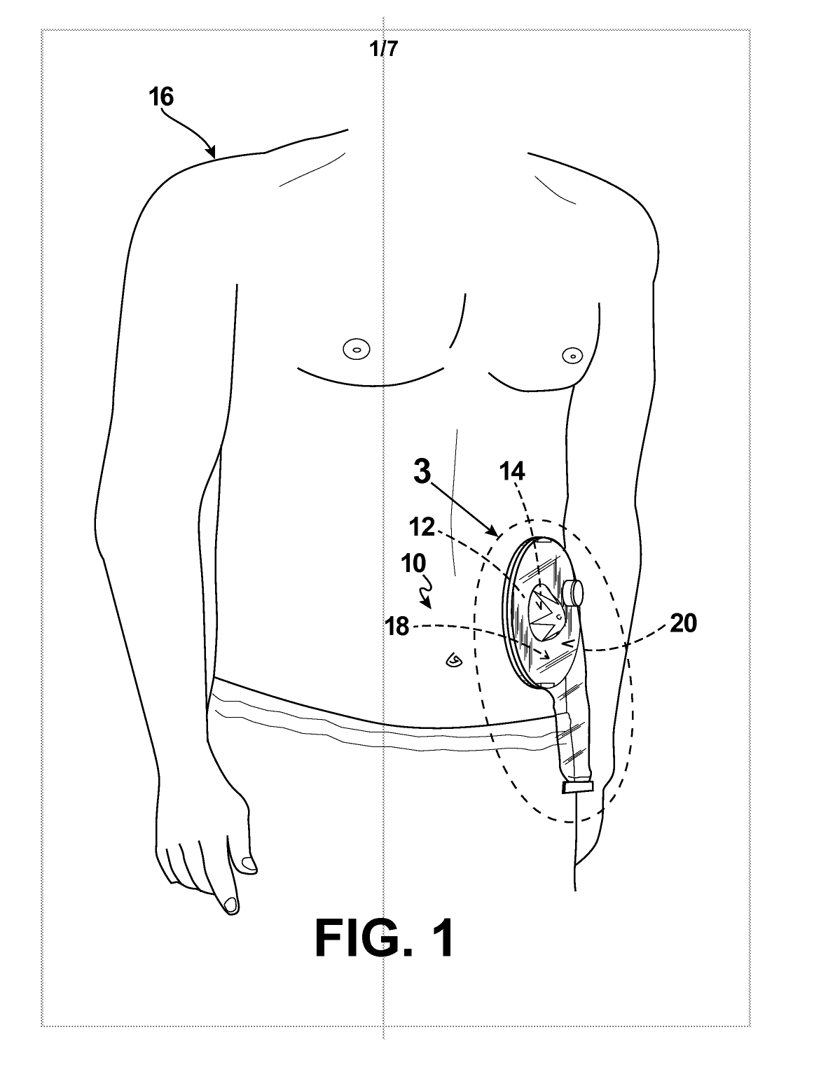

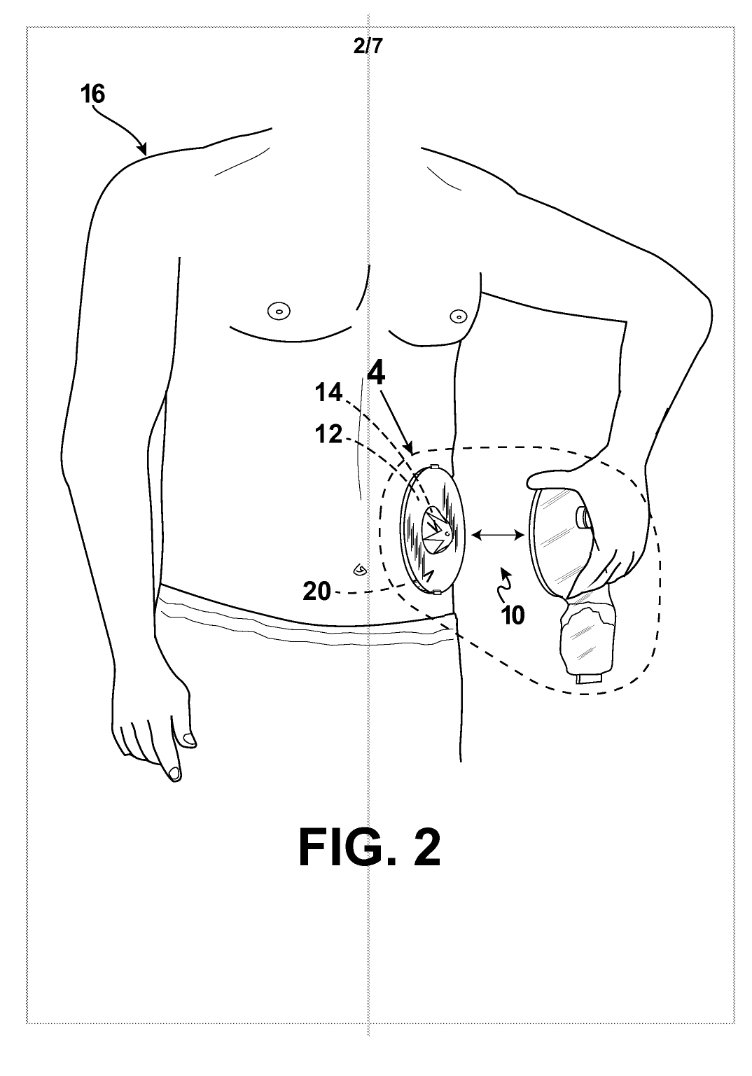

Referring now to the figures, in which like numerals indicate like parts, and particularly to FIGS. 1 and 2, which are, respectively, a diagrammatic perspective view of the ostomy device of the embodiments of the present invention fully attached to a site of a stoma of a patient, self-sealed to the stoma of the patient, and venting gas emanating from the stoma of the patient to prevent build-up of the gas that could detach the ostomy device from the site of the stoma of the patient and require rettachments that could irritate skin of the patient at the site of the stoma of the patient, and an exploded diagrammatic perspective view of the ostomy device of the embodiments of the present invention partially attached to a site of a stoma of a patient, self-sealed to the stoma of the patient, and venting gas emanating from the stoma of the patient to prevent build-up of the gas that could detach the ostomy device from the site of the stoma of the patient and require rettachments that could irritate skin of the patient at the site of the stoma of the patient, the ostomy device of the embodiments of the present invention is shown generally at 10 for attaching to a site 12 of a stoma 14 of a patient 16, for self-sealing to the stoma 14 of the patient 16, and for venting gas 18 emanating from the stoma 14 of the patient 16 to prevent build-up of the gas 18 that could detach the ostomy device 10 from the site 12 of the stoma 14 of the patient 16 and require rettachments that could irritate skin 20 of the patient 16 at the site 12 of the stoma 14 of the patient 16.

B. Overall Configuration of the Ostomy Device 10.

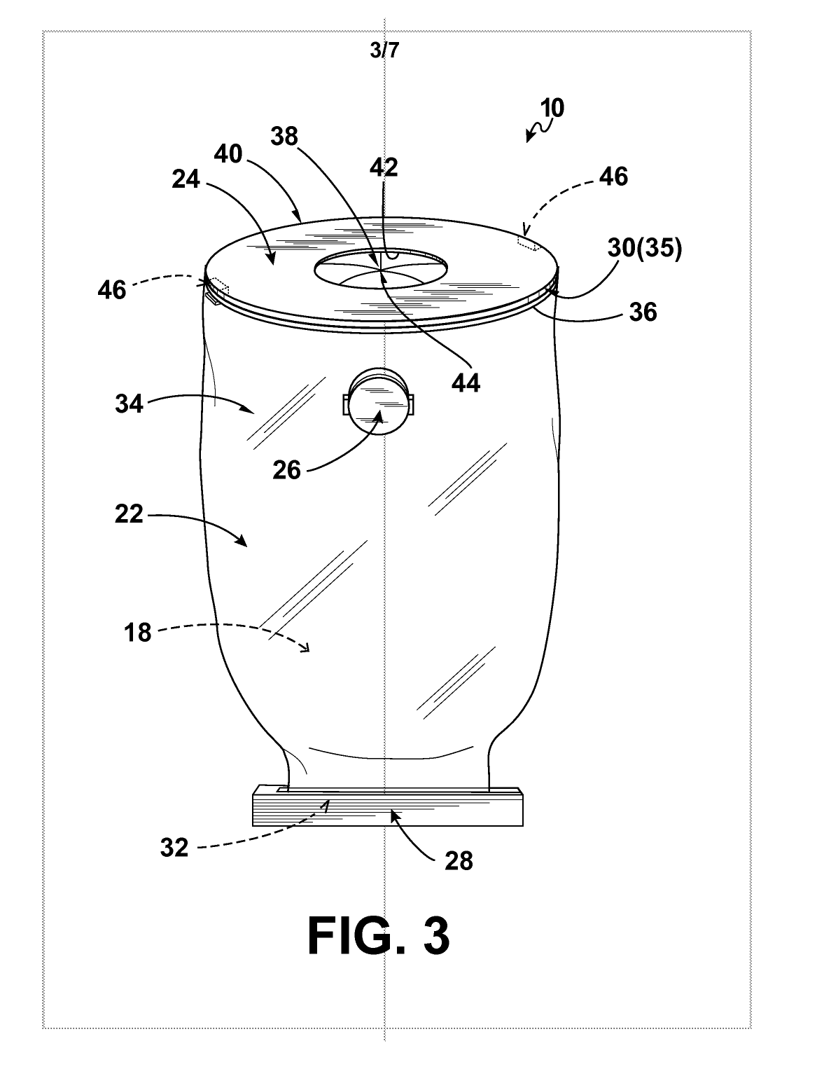

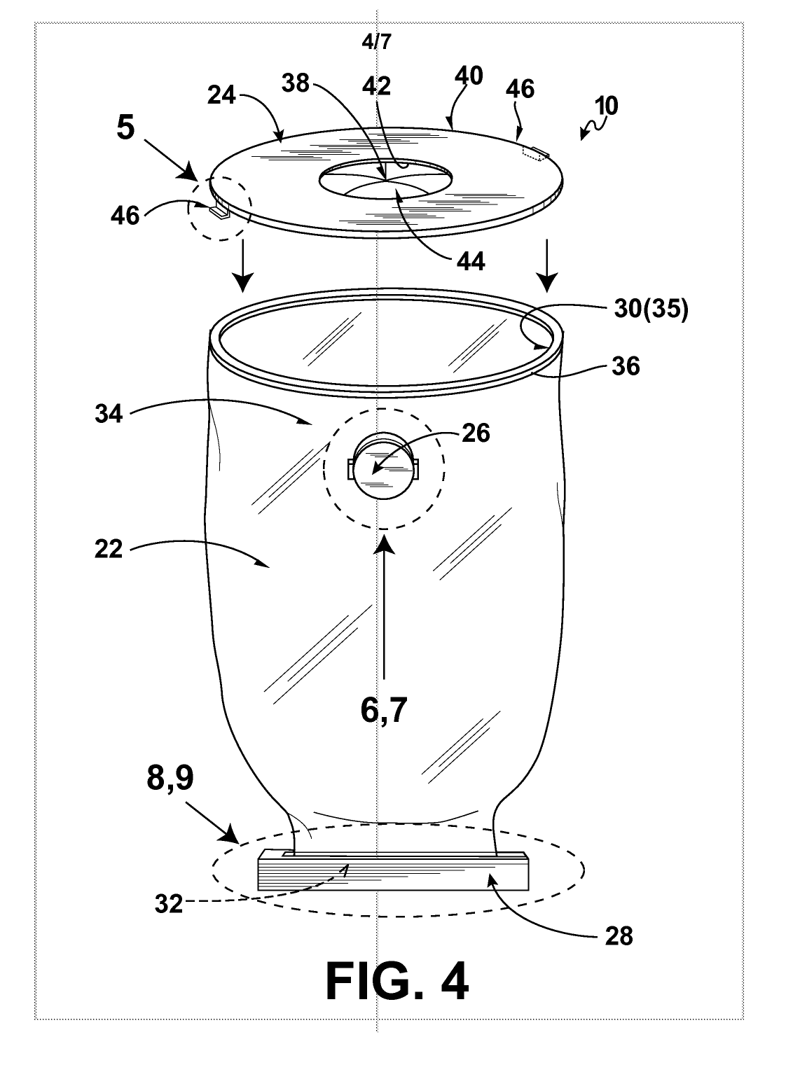

The overall configuration of the ostomy device 10 can best be seen in FIGS. 3 and 4, which are, respectively, an enlarged diagrammatic perspective view of the area generally enclosed by the dotted curve identified by ARROW 3 in FIG. 1 of the ostomy device of the embodiments of the present invention, and an enlarged and exploded diagrammatic perspective view of the area generally enclosed by the dotted curve identified by ARROW 4 in FIG. 2 of the ostomy device of the embodiments of the present invention, and as such, will be discussed with reference thereto.

The ostomy device 10 comprises a bag 22, a lid 24, and a vent 26. The lid 24 is replaceably attached to the bag 22, and is for attaching to the site 12 of the stoma 14 of the patient 16 and for self-sealing to the stoma 14 of the patient 16. The vent 26 is operatively connected to the bag 22, and is for venting the gas 18 emanating from the stoma 14 of the patient 16 to prevent the build-up of the gas 18 that could detach the ostomy device 10 from the site 12 of the stoma 14 of the patient 16 and require rettachments that could irritate the skin 20 of the patient 16 at the site 12 of the stoma 14 of the patient 16.

The ostomy device 10 further comprises a clip 28.

The clip 28 is replaceably attached to the bag 22, and is for allowing emptying of the bag 22 while the bag 22 is still attached to the site 12 of the stoma 14 of the patient 16.

C. Specific Configuration of the Bag 22.

The bag 22 has a wide and open top 30, a narrow and open bottom 32, and a wall 34.

The narrow and open bottom 32 of the bag 22 is narrower than the wide and open top 30 of the bag 22.

The wide and open top 30 of the bag 22 has a ring shape 35, and is defined by a rigid ring 36.

The ring shape 35 of the wide and open top 30 of the bag 22 is maintained by the rigid ring 36 of the bag 22.

D. Specific Configuration of the Lid 24.

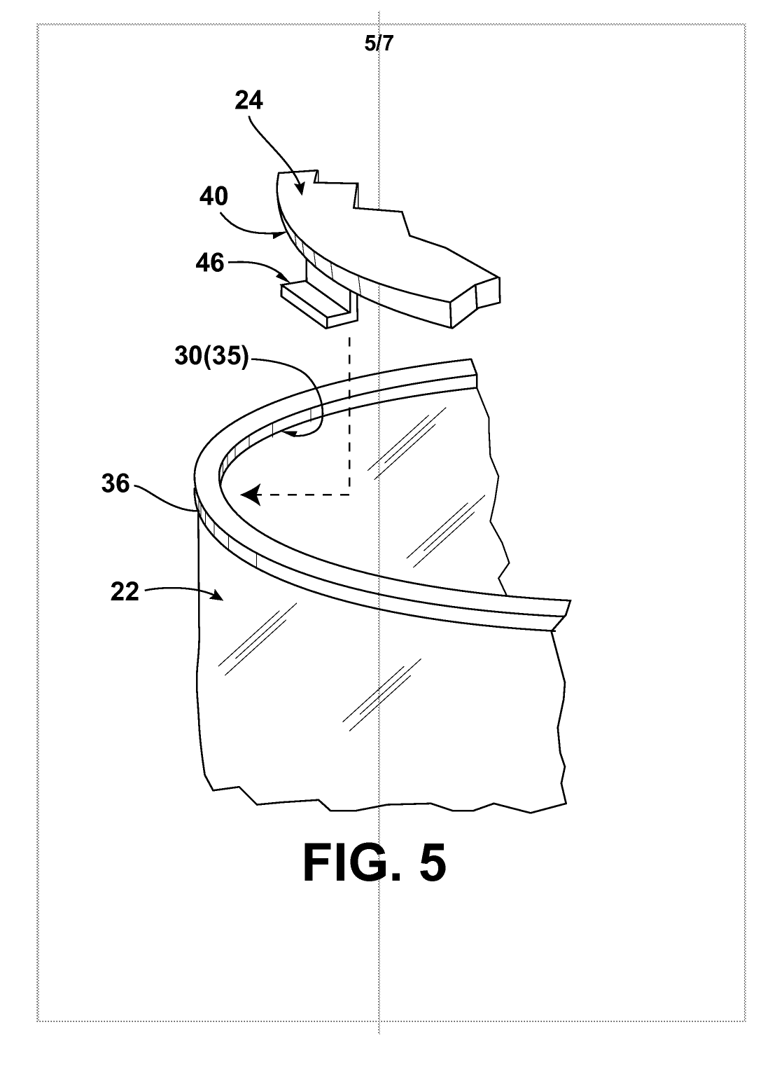

The specific configuration of the lid 24 can best be seen in FIGS. 3-5, which are, respectively, again, an enlarged diagrammatic perspective view of the area generally enclosed by the dotted curve identified by ARROW 3 in FIG. 1 of the ostomy device of the embodiments of the present invention, again, an enlarged and exploded diagrammatic perspective view of the area generally enclosed by the dotted curve identified by ARROW 4 in FIG. 2 of the ostomy device of the embodiments of the present invention, and an enlarged diagrammatic perspective view of the area generally enclosed by the dotted curve identified by ARROW 5 in FIG. 4 of a snap fastener of the osotomy device of the embodiments of the present invention replaceably attaching a snap-on flanged lid of the osotomy device of the embodiments of the present invention to a removable bag of the osotomy device of the embodiments of the present invention, and as such, will be discussed with reference thereto.

The lid 24 is disk-shaped, and has a center 38 and a perimeter 40.

The lid 24 further has a through bore 42.

The through bore 42 of the lid 24 is disposed at the center 38 of the lid, and has a size for receiving the stoma 14 of the patient 16.

The lid 24 further has a barrier ring 44.

The barrier ring 44 of the lid 24 fills the through bore 42 of the lid 24, and is expandable and retractable for self-sealing to the stoma 14 of the patient 16 when the lid 24 is attached to the site 12 of the stoma 14 of the patient 16.

The lid 24 further has a pair of L-shaped tabs 46.

The pair of L-shaped tabs 46 of the lid 24 depend from the perimeter 40 of the lid 24, and are diametrically opposed to each other.

The pair of L-shaped tabs 46 of the lid 24 replaceably and snappingly engage the rigid ring 36 of the bag 22 so as to allow the lid 24 to be snapped onto the bag 22 and form an air-tight seal therewith.

E. Specific Configuration of the Vent 26.

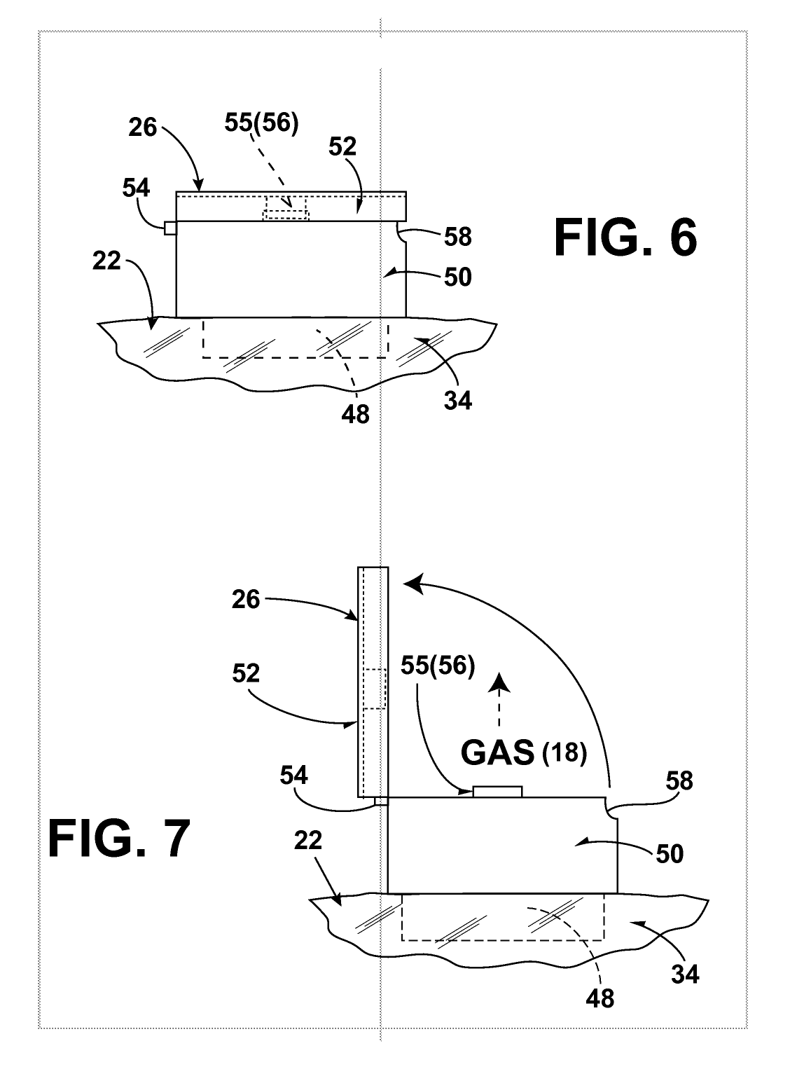

The specific configuration of the vent 26 can best be seen in FIGS. 6 and 7, which are, respectively, an enlarged diagrammatic elevational view taken generally in the direction of ARROW 6 in FIG. 4 of an openable vent of the osotomy device of the embodiments of the present invention in a closed position, and an enlarged diagrammatic elevational view taken generally in the direction of ARROW 7 in FIG. 4 of the openable vent of the osotomy device of the embodiments of the present invention in an open position, and as such, will be discussed with reference thereto.

The vent 26 comprises the wall 34 of the bag 22 having a through bore 48 extending therethrough.

The through bore 48 of the wall 34 of the bag 22 is disposed closer to the wide and open top 30 of the bag 22 than the narrow and open bottom 32 of the bag 22 since the gas 18 thermodynamically rises.

The vent 26 further comprises a collar 50.

The collar 50 of the vent 26 is attached to the wall 34 of the bag 22, and surrounds, and communicates with, the through bore 48 of the wall 34 of the bag 22.

The vent 26 further comprises a cap 52.

The cap 52 of the vent 26 is hingedly attached, by a hinge 54, to the collar 50 of the vent 26, and when closed, seals off the vent 26 from venting the gas 18, and when opened, vents the gas 18 emanating from the stoma 14 of the patient 16 to prevent the build-up of the gas 18 that could detach the ostomy device 10 from the site 12 of the stoma 14 of the patient 16 and require rettachments that could irritate the skin 20 of the patient 16 at the site 12 of the stoma 14 of the patient 16.

The vent 26 further comprises a snap closure 55.

The snap closure 55 of the vent 26 has a pair of cooperating portions 56.

The pair of cooperating portions 56 of the snap closure 55 of the vent 26 are disposed on the collar 50 of the vent 26 and on the cap 52 of the vent 26, respectively, so as to snappingly engage each other when the cap 52 of the vent 26 is closed onto the collar 50 of the vent 26.

The collar 50 of the vent 26 has a recess 58.

The recess 58 of the collar 50 of the vent 26 is disposed diametrically opposite to the hinge 54 of the vent 26, directly under the cap 52 of the vent 26 when the cap 52 of the vent 26 is closed, and is for facilitating grabbing the cap 52 of the vent 26 when the cap 52 of the vent 26 is to be opened.

F. Specific Configuration of the Clip 28.

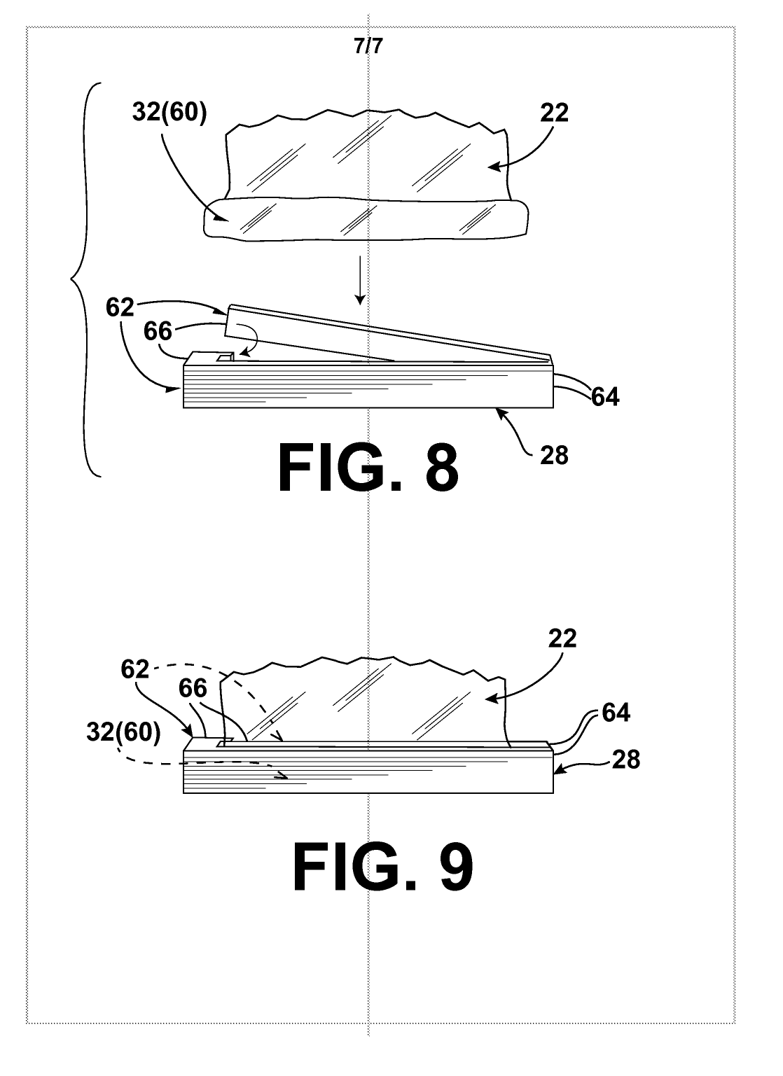

The specific configuration of the clip 28 can best be seen in FIGS. 8 and 9, which are, respectively, an enlarged and exploded diagrammatic perspective view of the area generally enclosed by the dotted curve identified by ARROW 8 in FIG. 4 of a reusable and leak-proof clip of the osotomy device of the embodiments of the present invention in an open position, and an enlarged and exploded diagrammatic perspective view of the area generally enclosed by the dotted curve identified by ARROW 9 in FIG. 4 of the reusable and leak-proof clip of the osotomy device of the embodiments of the present invention in a closed position, and as such, will be discussed with reference thereto.

The clip 28 is reusable and provides a leak-proof closure.

The clip 28 replaceably engages and selectively closes the narrow and open bottom 32 of the bag 22 after the narrow and open bottom 32 of the bag 22 has been folded onto itself so as to form a seal 60, and is for allowing the narrow and open bottom 32 of the bag 22 to be closed after the narrow and open bottom 32 of the bag 22 has been opened to empty the bag 22.

The clip 28 comprises a pair of cooperating portions 62.

The pair of cooperating portions 62 of the clip 28 are hinged together at associated ends 64 thereof and are snappingly engageable with each other at the other associated ends 66 thereof, and in doing so, captures and provides a leak-proof seal for the seal 60 of the narrow and open bottom 32 of the bag 22.

G. Impressions.

It will be understood that each of the elements described above or two or more together may also find a useful application in other types of constructions differing from the types described above.

While the embodiments of the present invention have been illustrated and described as embodied in an ostomy device for attaching to a site of a stoma of a patient, for self-sealing to the stoma of the patient, and for venting gas emanating from the stoma of the patient to prevent build-up of the gas that could detach the ostomy device from the site of the stoma of the patient and require rettachments that could irritate skin of the patient at the site of the stoma of the patient, however, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. An ostomy device for attaching to a site of a stoma of a patient, for self-sealing to the stoma of the patient, and for venting gas emanating from the stoma of the patient to prevent build-up of the gas that could detach said ostomy device from the site of the stoma of the patient and require rettachments that could irritate skin of the patient at the site of the stoma of the patient, said ostomy device comprising:
a) a bag;
b) a lid; and
c) a vent;
wherein said lid is replaceably attached to said bag;
wherein said lid is for attaching to the site of the stoma of the patient;
wherein said lid is for self-sealing to the stoma of the patient;
wherein said vent is operatively connected to said bag; and
wherein said vent is for venting the gas emanating from the stoma of the patient to prevent the build-up of the gas that could detach said ostomy device from the site of the stoma of the patient and require rettachments that could irritate the skin of the patient at the site of the stoma of the patient.

2. The ostomy device of claim 1, further comprising a clip;
wherein said clip is replaceably attached to said bag; and
wherein said clip is for allowing emptying of said bag while said bag is still attached to the site of the stoma of the patient.

3. The ostomy device of claim 2, wherein said bag has a wide and open top.

4. The ostomy device of claim 3, wherein said bag has a narrow and open bottom.

5. The ostomy device of claim 4, wherein said narrow and open bottom of said bag is narrower than said wide and open top of said bag.

6. The ostomy device of claim 3, wherein said wide and open top of said bag is defined by a rigid ring.

7. The ostomy device of claim 6, wherein said wide and open top of said bag has a ring shape; and
wherein said ring shape of said wide and open top of said bag is maintained by said rigid ring of said bag.

8. The ostomy device of claim 1, wherein said lid is disk-shaped.

9. The ostomy device of claim 1, wherein said lid has a through bore.

10. The ostomy device of claim 9, wherein said lid has a center;
wherein said through bore of said lid is disposed at said center of said lid; and
wherein said through bore of said lid has a size for receiving the stoma of the patient.

11. The ostomy device of claim 9, wherein said lid has a barrier ring.

12. The ostomy device of claim 11, wherein said barrier ring of said lid fills said through bore of said lid; and
wherein said barrier ring of said lid is expandable and retractable for self-sealing to the stoma of the patient when said lid is attached to the site of the stoma of the patient.

13. The ostomy device of claim 6, wherein said lid has a pair of L-shaped tabs.

14. The ostomy device of claim 13, wherein said lid has a perimeter; and
wherein pair of L-shaped tabs of said lid depend from said perimeter of said lid.

15. The ostomy device of claim 13, wherein said pair of L-shaped tabs of said lid are diametrically opposed to each other.

16. The ostomy device of claim 13, wherein said pair of L-shaped tabs of said lid replaceably and snappingly engage said rigid ring of said bag so as to allow said lid to be snapped onto said bag and form an air-tight seal therewith.

17. The ostomy device of claim 4, wherein said bag has a wall; and
wherein said vent comprises said wall of said bag having a through bore extending therethrough.

18. The ostomy device of claim 17, wherein said through bore of said wall of said bag is disposed closer to said wide and open top of said bag than said narrow and open bottom of said bag since the gas thermodynamically rises.

19. The ostomy device of claim 17, wherein said vent comprises a collar.

20. The ostomy device of claim 19, wherein said collar of said vent is attached to said wall of said bag.

21. The ostomy device of claim 19, wherein said collar of said vent surrounds said through bore of said wall of said bag.

22. The ostomy device of claim 19, wherein said collar of said vent communicates with said through bore of said wall of said bag.

23. The ostomy device of claim 19, wherein said vent comprises a cap.

24. The ostomy device of claim 23, wherein said cap of said vent is attached to said collar of said vent, and when closed, seals off said vent from venting the gas, and when opened, vents the gas emanating from the stoma of the patient to prevent the build-up of the gas that could detach said ostomy device from the site of the stoma of the patient and require rettachments that could irritate the skin of the patient at the site of the stoma of the patient.

25. The ostomy device of claim 23, wherein said cap of said vent is hingedly attached by a hinge to said collar of said vent.

26. The ostomy device of claim 25, wherein said vent comprises a snap closure.

27. The ostomy device of claim 26, wherein said snap closure of said vent has a pair of cooperating portions.

28. The ostomy device of claim 27, wherein said pair of cooperating portions of said snap closure of said vent are disposed on said collar of said vent and on said cap of said vent, respectively, so as to snappingly engage each other when said cap of said vent is closed onto said collar of said vent.

29. The ostomy device of claim 23, wherein said collar of said vent has a recess; and
wherein said recess of said collar of said vent is for facilitating grabbing said cap of said vent when said cap of said vent is to be opened.

30. The ostomy device of claim 29, wherein said recess of said collar of said vent is disposed diametrically opposite to said hinge of said vent.

31. The ostomy device of claim 29, wherein said recess of said collar of said vent is disposed directly under said cap of said vent when said cap of said vent is closed.

32. The ostomy device of claim 2, wherein said clip is reusable.

33. The ostomy device of claim 2, wherein said clip provides a leak-proof closure.

34. The ostomy device of claim 4, wherein said clip replaceably engages said narrow and open bottom of said bag after said narrow and open bottom of said bag has been folded onto itself so as to form a seal; and
wherein said clip is for allowing said narrow and open bottom of said bag to be closed after said narrow and open bottom of said bag has been opened to empty said bag.

35. The ostomy device of claim 4, wherein said clip selectively closes said narrow and open bottom of said bag after said narrow and open bottom of said bag has been folded onto itself so as to form a seal.

36. The ostomy device of claim 34, wherein said clip comprises a pair of cooperating portions.

37. The ostomy device of claim 36, wherein said pair of cooperating portions of said clip are hinged together at associated ends thereof and are snappingly engageable with each other at the other associated ends thereof, and in doing so, captures and provides a leak-proof seal for said seal of said narrow and open bottom of said bag.
